(19) United States
(12) Patent Application Publication
(10) Pub. No.: US 2010/0004174 A1
(43) Pub. Date: Jan. 7, 2010

(54) USE OF THYMOSIN ALPHA 1 FOR THE PREPARATION OF A MEDICAMENT FOR THE PREVENTION AND TREATMENT OF AUTOIMMUNE DISEASES

(75) Inventors: Luigina Romani, Perugia (IT); Francesco Bistoni, Perugia (IT); Enrico Garaci, Roma (IT); Guido Rasi, Roma (IT); Paola Sinbaldi Vallebona, Roma (IT)

Correspondence Address: ARENT FOX LLP, 1050 CONNECTICUT AVENUE, N.W., SUITE 400, WASHINGTON, DC, 20036, US

(73) Assignee: SIGMA-TAU INDUSTRIE FARMACEUTICHE, Pomezia (IT)

(21) Appl. No.: 12/446,670

(22) PCT Filed: Sep. 27, 2007
(86) PCT No.: PCT/IT2007/000677

§ 371 Date: Jun. 25, 2009

(30) Foreign Application Priority Data
Oct. 27, 2006 (IT) RM2006A000584

(51) Int. Cl. A61K 38/22; A61P 37/00; A61P 29/00

(52) U.S. Cl. 514/12

(57) ABSTRACT

The invention concerns the use of thymosin alpha 1 for the preparation of a medicament for the prevention and treatment of autoimmune diseases such as multiple sclerosis, and inflammatory bowel diseases such as Crohn's disease or ulcerative colitis.

The present invention concerns the use of thymosin alpha 1 for the preparation of a medicament for the prevention and treatment of autoimmune diseases. In particular, the invention refers to the use of thymosin alpha 1 for the preparation of a medicament for the prevention and treatment of autoimmune diseases such as multiple sclerosis (MS) and inflammatory bowel diseases such as Crohn's disease or ulcerative colitis.

The fight against inflammatory-based neurological diseases is one of the main scientific, social and economic objectives at world level. In the last few years it has emerged that, in a considerable number of pathologies of the central nervous system (CNS), such as MS, infective and post-infective encephalomyelitis and encephalitis, the neurological complications of autoimmune diseases, Rasmussen's encephalitis, ischaemia and traumatic brain damage are due to an inflammatory process closely associated with neuronal and axonal damage. Neurodegeneration is the main cause of permanent neurological deficiencies. Neuroinflammatory illnesses involve a high economic cost for society, since they are associated with serious disability such as sensory-motor deficiencies, a worsening of cognitive functioning, incontinence and behavioural problems. Multiple sclerosis (MS) is the main cause of neurological disability in young adults in the western world. MS is a chronic inflammatory disease of the CNS with an unknown etiology that has both genetic and environmental underlying factors. As already mentioned, it is the main cause of neurological disability in young adults in the western world, with the highest rates found in central and northern Europe (>30 cases for every 100,000 inhabitants). In most patients, the disease arises with episodes of neurological dysfunction followed by complete or partial remission (relapsing-remittent MS) and then later presents a progressive trend with growing disability (secondary progressive MS). Some patients immediately show a progressive trend (primary progressive MS) while an acute trend of the disease is rarer. Pathologically, MS is characterised by the presence of damage in the white matter ascertained by magnetic resonance imaging. Inflammatory infiltrates, loss of myelin (the lipid-rich membrane covering axons) and oligodendrocytes (myelin producing cells), axon damage and reactive gliosis have all been seen in the lesions, or plaques. Recently, more detailed neuropathological studies and the application of advanced magnetic resonance imaging techniques have shown the existence of more widespread inflammatory damage in the white matter and the presence of lesions in the grey matter of the cerebral cortex, that are associated with neurodegenerative processes and predominate in the progressive forms of the disease. Despite the considerable number of clinical and experimental studies, the complex immunopathological mechanisms underlying myelin and neuron damage are still largely unknown. The autoimmune diseases arise when the immune system, which normally defends the body from diseases and infections, attacks itself and it can strike many parts of the body such as the nerves or muscles, as well as cause significant and chronic morbilities and invalidities. Most of what is known of this disease derives from the study of experimental autoimmune encephalitis (EAE), an animal de-myelination model mimicking what happens in man. In practice, the animals are sensibilised towards certain proteins of myelin to cause symptoms like the ones of multiple sclerosis, and the experimental therapies are then tested on them in order to assess their effectiveness. Cell therapy uses a certain type of cell, and namely dentiritic ones, which—when injected into the animals affected by these disorders—generate T regulator cells (Treg) responsible for maintaining immunitary tolerance. Recent studies have shown that dendritic cells cause the growth of new Treg cells in the treated animals and that these cells specifically neutralise the immunitary cells that attack the myelin covering the nerves. The treatment was effective also with in-vitro generated Treg cells. Recent studies have shown that thymosin alpha 1, a thymic hormone that is already widely used in the clinical art for various pathologies (Goldstein, A. L. Badamchian, M. Thymosins: chemistry and biological properties in health and disease. Expert Opin Biol Ther. 2004, 4: 559-73), can induce Treg in vitro for a selective action on dendritic cells (Romani L, Bistoni F, Perruccio K, Montagnoli C, Gaziano R, Bozza S, Bonifazi P, Bistoni G, Rasi G, Velardi A, Fallarino F, Garaci E, Puccetti P. Thymosin alpha 1 activates dendritic cell tryptophan catabolism and establishes a regulatory environment for balance of inflammation and tolerance. Blood. 2006 Jun. 1; [Epub ahead of print]).

Inflammatory bowel disease (IBD) may involve both the small and large intestines. Crohn's disease and ulcerative colitis are the most known forms of IBD and both belong to the category of idiopathic inflammatory bowel diseases because their etiology is unknown. The pathological tests are not generally specific, even if they may suggest a particular form of IBD. “Active” IBD is characterised by acute inflammation. “Chronic” IBD is characterised by architectural changes of distortion and gouging of the bowel crypts. The crypt abscesses (consisting of neutrophils activated in the crypt lumen) can be present in many forms of IBD.

Ulcerative colitis (UC) is a predominantly distal mucous problem. The rectum is virtually always involved. The etiology of UC is unknown. UC is more common in Caucasians, women and young adults (peak of incidence around 20-25 years of age). Clinical results include diarrhea, with or without tenesmus. Patients suffering from prolonged UC are at great risk of developing malignant neoplasies. Colon biopsy can be used to ascertain dysplasia, a neoplastic change in the mucosa which means a high probability of malignant neoplasia. Patients with UC are also at risk of developing hepatic disorders including sclerosing cholangitis and carcinoma of the biliary ducts.

Crohn's disease may affect any part of the bowels, but most often involves the tenuous and distal parts. The inflammation is generally transmural and may produce something like a small ulcer above the lymphoid follicle or a deep ulcer in the transmural gash and in chronic inflammation. A third of the cases have granulomatosis in regions beyond the colon such as the linfonodes and liver. Transmural inflammation leads to the development of fistula between the bowels and other structures. The inflammation is generally segmental. The etiology is unknown, although infective and immunological mechanisms have been suggested. There is a bimodal incidence and an increased incidence in women and in Caucasians. The clinical manifestations vary and can include diarrhea, fever and pain, as well as non-bowel manifestations due to arthritis, uveitis, erythema nodosum and ankylosing spondylitis.

Comparison of ulcerative colitis and Crohn's disease

| Characteristic | Ulcerative colitis | Crohn's disease |
|---|---|---|
| Distribution | Widespread, distal | Segmental or widespread |
| | predominance | predominance, often proximal |
| Rectum | Always involved | Often spared |
| Microscopic | Widespread | Often focal |
| distribution | | |
| Extent of | Mucous | Transmural |
| inflammation | | |
| Breast traits and | Absent | Often present |
| fistula | | |
| Constriction | Absent | Often present |
| Granuloma | Absent | Often present |

The infective causes of IBD generally have a more acute onset and a shorter duration. The bacterial organisms that can cause IBD include Shigella, Salmonelle, Capylobacter and a certain number of E. coli. Bacteria are a common cause of self-limiting colitis, without chronic changes. The viral etiologies include Norwalk Virus, Rotavirus as well as cytomegalovirus. Other causes include chlamydial infection and amebiasis. IBD linked to the use of antibiotics can be secondary to therapy using broad range antibiotics that lead to the excessive growth of Clostridium difficile or of other organisms such as Candida albicans. IBD may also be secondary to ischaemia.

Pharmacological treatment is the main way to alleviate the symptoms of both ulcerative colitis and Crohn's disease. There has been much progress in the development of medicaments for the treatment of IBD. These include:
Anti-inflammatory drugs, used in order to decrease the inflammation caused by the disease.
Immunosuppressive agents hindering the immune system's attack of the tissues of its own body thereby causing the viscious circle of inflammation.

If a person with IBD does not respond to these drugs, then surgery is required. However, the surgical procedures for UC and Crohn's disease are very different. In Crohn's disease the physician must try as much as possible to avoid surgery because of the recurrent nature of the disease. Moreover, an aggressive surgical approach to Crohn's disease is thought to cause further complications such as short bowel syndrome, which creates problems in growth and the reduced capability to take in nutrients. As regards UC, removing the colon (large bowel) may be necessary along with the surgical procedure called ileoanal anastomosis (also called ileoanal pull-through) in which the physician creates a pocket in the small bowel to collect the faeces in the pelvis. This enables the faeces to pass through the anus.

Recent findings indicate that IBD may be the result of a immunological dysregulation involving inflammatory effectors and regulatory cells (Treg). In this regard, in experimental models of IBD, three scenarios may be identified that may be correlated to the IBD outcome:
a. Balanced effectory and regulatory responses=control of the inflammation and no signs of IBD.
b. A prevalence of the effector response on the regulatory one=inflammation and IBD.
c. No regulation=uncontrolled inflammation and severe IBD.

The number of products under study for the treatment of IBD increased from 3 products and 1 target in 1993 to over 30 products and more than 10 targets in 2005. Colitis from TNBS (see below) serves as a useful preclinical model to test innovative treatments for Crohn's disease. These include: the application of 5-aminosalicylic acid and leukotriene antagonist in the colon, systemic treatments with prednisolone derivatives releasing nitric oxide, antagonists of chemokines and anti-adherence molecules. Anti-TNF approaches that are promising, from a clinical standpoint, in subgroups of patients with Crohn's disease also show beneficial effects in chronic TNBS colitis. The number of products being developed and studied in the pathogenesis of IBD emphasises the need to increase clinical research efforts on IBD. Recent studies have shown that thymosin alpha 1, a thymic hormone already widely used in the clinical art for various pathologies, can cause Treg in vitro for a selective action on dendritic cells.

To date, autoimmune diseases are treated with immunosuppressive or anti-inflammatory drugs such as corticosteroids. However, these drugs can have, even serious, side-effects such as immunosuppression and hyperglycemia.

In view of the above, there is thus an evident need for new drugs for the treatment and prevention of autoimmune diseases that do not present the disadvantages of known treatments.

The authors of the present invention have now found that thymosin alpha 1 is able to prevent and effectively treat autoimmune diseases without causing any toxic effect for the body.

Thymosin alpha 1 (Tα1), a thymic peptide found in nature, is well known for the treatment of certain viral infections both as a monotherapy and in association with IFN-α, and as an immunitary adjuvant

(Goldstein A. et al., 2004 Expert Opin.Biol. Ther. 4:559-573). Other therapeutic indications of thymosin alpha 1 are also known, such as in the treatment of immunodeficiency, tumours and AIDS. It has recently been shown that Tα1 modulates the functioning of dendritic cells (DC) through TLR9, thereby acting as an endogenous regulator of the innate and adaptive immune systems (Romani L. et al., 2004, Blood 103:4232-4239).

Thymosin alpha 1 is also known as a modulator of the biological response for the treatment of certain viral infections in combination with INF-α, and as an immune adjuvant (Goldstein A. et al., 2004 Expert Opin. Biol. Ther. 4:559-573).

The specific object of the present invention is thus the use of thymosin alpha 1 for the preparation of a medicament for the prevention and treatment of autoimmune diseases, preferably multiple sclerosis and inflammatory viscera diseases such as inflammatory bowel diseases, for example ulcerative colitis and Crohn's disease.

The present invention will now be described for illustration purposes, but is not limited to these examples, according to its preferred embodiments and with particular reference to the figures in the attached drawings, wherein:

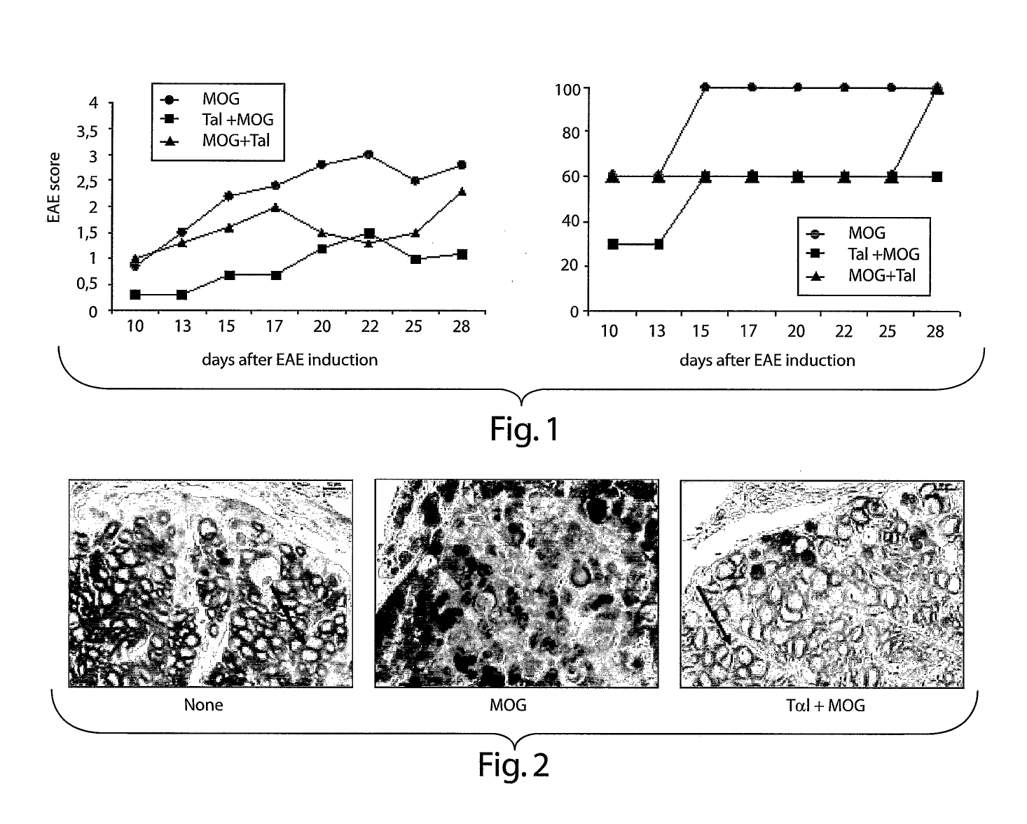

FIG. 1 shows the average clinical score of mice (n=5) against time (days) with regard to immunisation with the MOG peptide.

FIG. 2 shows the histological analysis of semifine spinal chord sections coloured with osmium tetroxide and highlights areas of demyelination in the mice treated with MOG, while there is no significant sign of demyelination in the mice treated with thymosin alpha 1. The black arrow indicates the integrity of the myelin sheath.

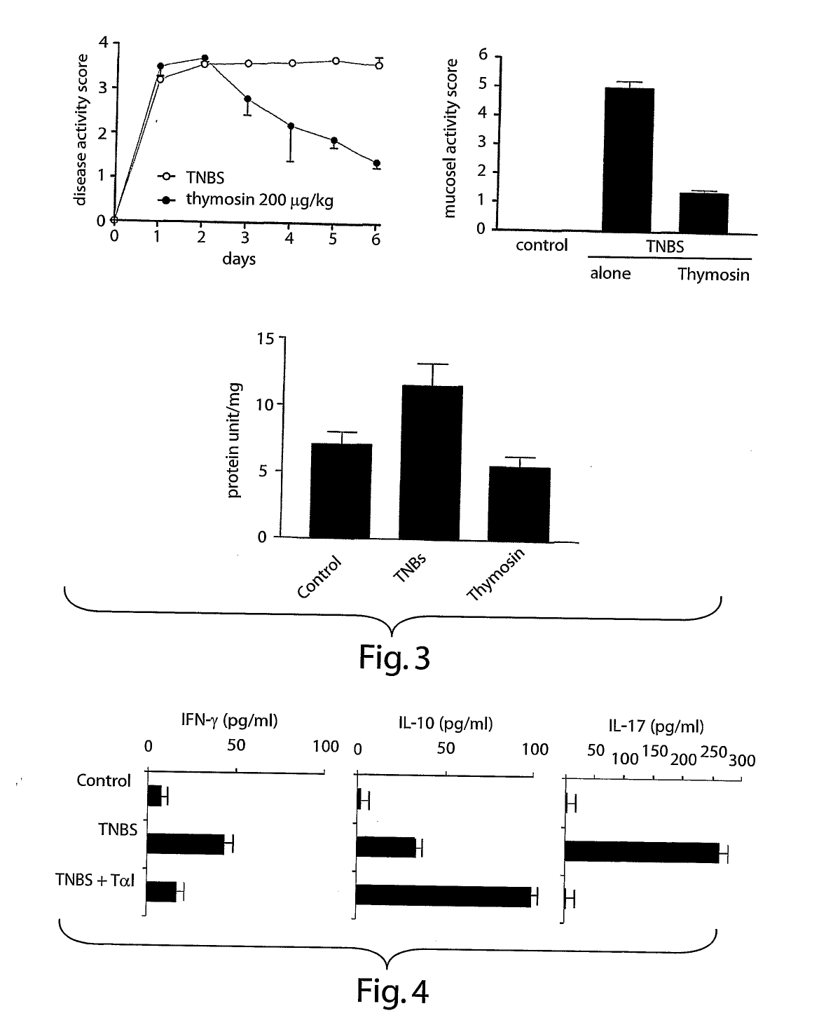

FIG. 3 shows the effect of thymosin alpha 1 on the severity of the induced colitis, measured in terms of DAS, MAS and MPO. The results show a drastic reduction of all the parameters associated and associable to the presence of frank colitis by thymosin alpha 1. The control mice did not receive any treatment.

FIG. 4 shows the cytokine production of CD4⁺CD²⁵+ isolated from the lamina of control animals, with colitis (TNBS) or treated with thymosin alpha 1 (TNBS+Tα1).

### EXAMPLE 1

### Evaluation of the Efficacy of Thymosin Alpha 1 in EAE

To this end, an EAE model was used envisaging the induction of disease by administering a peptide deriving from myelin in susceptible animals. The anima were treated with thymosin alpha 1, whose efficiency was evaluated according to clinical and histological parameters.

Methodology

EAE Induction

EAE is induced in C57BL6 mice by injecting 100 microlitres of emulsion consisting of 100 microgrammes of MOG35-55 (a peptide fragment of a myelinic glycoprotein) in complete Freund adjuvant (CFA, 4 microgrammes/ml) at the base of the tail. EAE induction is favoured by the intraperitoneal administration of the pertoxic toxin at a dosage of 200 ng/mouse the day of immunisation and three days later. The treatment with thymosin alpha 1 (200 ng/Kg) is carried out via intraperitoneal injection according to two experimental designs. In the first case, thymosin is administered starting from the day of MOG administration and for three consecutive days thereafter (Tα1+MOG). In the second case, thymosin is administered starting from the tenth day after MOG administration and for three consecutive days thereafter (MOG+Tα1).

Evaluation of EAE

The mice were clinically evaluated daily according to the following parameters: 0, normal; 1, flacid tail or weakness of hind legs; 2, flacid tail associated with weakness of hind legs; 3, partial paralysis of hind legs; 4, total paralysis of hind legs; 5, moribund state. The histological analysis of the semifine sections of the spinal chord was carried out by colouring with osmium tetroxide.

Results

The results (FIG. 1) show that thymosin alpha 1 is able to significantly induce both the gravity (score) and incidence of EAE in mice treated with MOG. The effect is particularly evident with thymosin administered both concomitantly with MOG sensibilization and later.

FIG. 2 shows the histological analysis of the semifine sections of the spinal chord coloured with osmium tetroxide and highlights areas of de-myelination in the mice treated with MOG, while no significant sign of de-myelination is found in the mice treated with thymosin alpha 1. The black arrow indicates the integrity of the myelin sheath. These results indicate that thymosin alpha 1 has a protective effect on EAE incidence as well as on its neuropathological severity.

### EXAMPLE 2

Evaluation of the efficacy of thymosin alpha 1 in an experimental model of IBD (Fiorucci S, Antonelli E, Distrutti E, Del Soldato P, Flower R J, Clark M J, Morelli A, Perretti M, Ignarro L J. NCX-1015, a nitric-oxide derivative of prednisolone, enhances regulatory T cells in the lamina propria and protects against 2,4,6-trinitrobenzene sulfonic acid-induced colitis in mice. Proc Natl Acad Sci U S A. 2002, 99:15770-5).

To this end, an IBD model was used envisaging the induction of the disease by intracolonic administration of a haptenised derivative of trinitrobenzene sulfonic acid (TNBS). The animals were treated with thymosin alpha 1, whose efficacy was evaluated according to clinical, histological and immunological parameters.

Methodology

Models of Colitis

The application of hapten TNBS in the colon causes acute and chronic colitis in the rodents (Fiorucci S, Antonelli E, Distrutti E, Del Soldato P, Flower R J, Clark M J, Morelli A, Perretti M, Ignarro L J. NCX-1015, a nitric-oxide derivative of prednisolone, enhances regulatory T cells in the lamina propria and protects against 2,4,6-trinitrobenzene sulfonic acid-induced colitis in mice. Proc Natl Acad Sci U S A. 2002, 99:15770-5).

The mucosal inflammation in colitis from TNBS has a neutrophilic infiltrate, but also includes the affluence of macrophages and monocytes as well as T lymphocyte activation (Th1). The histopathological characteristics are similar to Crohn's disease in man, to the transmural inflammation, granuloma, ulcers and skip lesions (patches of ulceration separated by patches of normal mucosa) (Morris G P, Beck P L, Herridge M S, Depew W T, Szewczuk M R, Wallace J L. Hapten-induced model of chronic inflammation and ulceration in the rat colon. Gastroenterology. 1989, 96:795-803).

The animals were monitored daily for any onset of diarrhea, loss of body weight, blood in the faeces and to check their survival (correlated with the degree of activity of the disease, DAS). At the end of the experiment, the surviving mice were sacrificed, their colon dissected and the microscopic damage was evaluated (called mucosal activity score—MAS). The tissue segments were then used in order to measure the Myeloperoxidase (MPO) activity (an inflammation index).

Microscopic Degree of Colitis

The sectioned colons were examined under a microscope (×5) and classified according to their microscopic lesions on a scale from 0 to 10 based on criteria reflecting the inflammation, such as hyperemia, bowel thickening, and extension of ulcers.

MPO assays. Neutrophil infiltration in the colon was monitored by measuring MPO activity by means of a spectrophotometric assay with trimethylbenzidine (TMB) as a substrate according to a previously published method (Fiorucci S, Antonelli E, Distrutti E, Del Soldato P, Flower R J, Clark M J, Morelli A, Perretti M, Ignarro L J. NCX-1015, a nitric-oxide derivative of prednisolone, enhances regulatory T cells in the lamina propria and protects against 2,4,6-trinitrobenzene sulfonic acid-induced colitis in mice. Proc Natl Acad Sci U S A. 2002, 99:15770-5). The activity is expressed in U per mg of protein.

Treatment with thymosin alpha 1. Tα1 (purchased from Sigma, St. Louis, Mo., USA; product no. T3410; molecular formula C₁₂₉H₂₁₅N₃₃O₅₅) and the scrambled peptide were provided in the form of sterile dried powders. The powders were reconstituted in sterile water (endotoxin levels were <0.03 pg/ml, by standard Limulus lysate assay). Thymosin alpha 1 was administered at a dosage of 200 microgrammi/kg by intraperitoneal injection for 6 consecutive days starting from the day of TNBS colitis induction.

Cytokine determination. This was carried out via specific ELISA tests (ELISA kit, R&D Systems Inc, Minneapolis, Minn.) on the supernatants of lymphocyte cell cultures of CD4⁺CD²⁵+ phenotype isolated from the lamina propria and stimulated in vitro for 48 h with antibodies directed against CD3 and CD28 molecules expressed on the lymphocytes (PharMingen, BD, Palo Alto, Calif.).

All the inflammatory and cytokine assessments were carried out the day after the end of treatment with thymosin alpha 1.

FIG. 3 shows the effect of thymosin alpha 1 on the severity of the induced colitis, measured in terms of DAS, MAS and MPO. The results show a drastic reduction of all the associated and associable parameters in the presence of frank colitis by thymosin alpha 1. The control mice received no treatment of any kind.

FIG. 4 shows the cytokine production of CD4⁺CD25⁺ isolated from lamina propria of control animals, animals with colitis (TNBS) or those treated with thymosin alpha 1 (TNBS+Tα1). The results show a clear increase in pro-inflammatory cytokines such as IFN-γ and IL-17, and a non-significant production of anti-inflamatory IL-10 in the animals with colitis. The inflammatory/anti-inflammatory cytokine production pattern was significantly disrupted by thymosin treatment, there being a clear prevalence of IL-10 production.

These results show that thymosin alpha 1 has a protective effect against the severity of IBD in experimental models of colitis.

1. Use of thymosin alpha 1 for the preparation of a medicament for the prevention and treatment of autoimmune diseases.

2. Use according to claim 1, wherein the autoimmune diseases are multiple sclerosis or inflammatory viscera diseases.

3. Use according to claim 2, wherein the inflammatory viscera diseases are specifically inflammatory bowel diseases.

4. Use according to claim 3, wherein the inflammatory bowel diseases are ulcerative colitis or Crohn's disease.
